(19) United States
(12) Patent Application Publication
(10) Pub. No.: US 2007/0239157 A1
(43) Pub. Date: Oct. 11, 2007

(54) Ancillary Assembly for Implanting a Knee Prosthesis

(76) Inventor: Francis Guillaume, Sanvignes Les Mines (FR)

Correspondence Address: Ralph A. Dowell of DOWELL & DOWELL P.C., 2111 Eisenhower Ave, Suite 406, Alexandria, VA, 22314, US

(21) Appl. No.: 10/594,062

(22) PCT Filed: Mar. 25, 2005
(86) PCT No.: PCT/FR05/00726

§ 371 Date: May 15, 2007

(30) Foreign Application Priority Data
Mar. 26, 2004 (FR) 04 03156

(51) Int. Cl. A61B 17/58

(52) U.S. Cl. 606/60

(57) ABSTRACT

The assembly comprises a first instrument (10) for distracting the knee and comprising firstly two branches (13, 14) movable relative to each other and provided at their distal ends respectively with means (26) for pressing against the tibia and means (28) for pressing against the femur, and secondly means (16) for moving the distal ends of the branches mutually apart. The distraction instrument (10) further comprises a device (12) for acting on the medial side and on the lateral side of the femur to locate the positions where at least one pair of extramedullary pins are to be implanted in respective directions lying in a common plane substantially parallel to a plane tibial bearing surface defined by the means (26) for bearing against the tibia, said plane being situated at an adjustable distance from said tibial bearing surface. The invention is applicable to implanting a knee prosthesis, in particular a prosthesis including a moving plate.

The present invention relates to a set of instruments for implanting a knee prosthesis, in particular a so-called “moving plate” prosthesis.

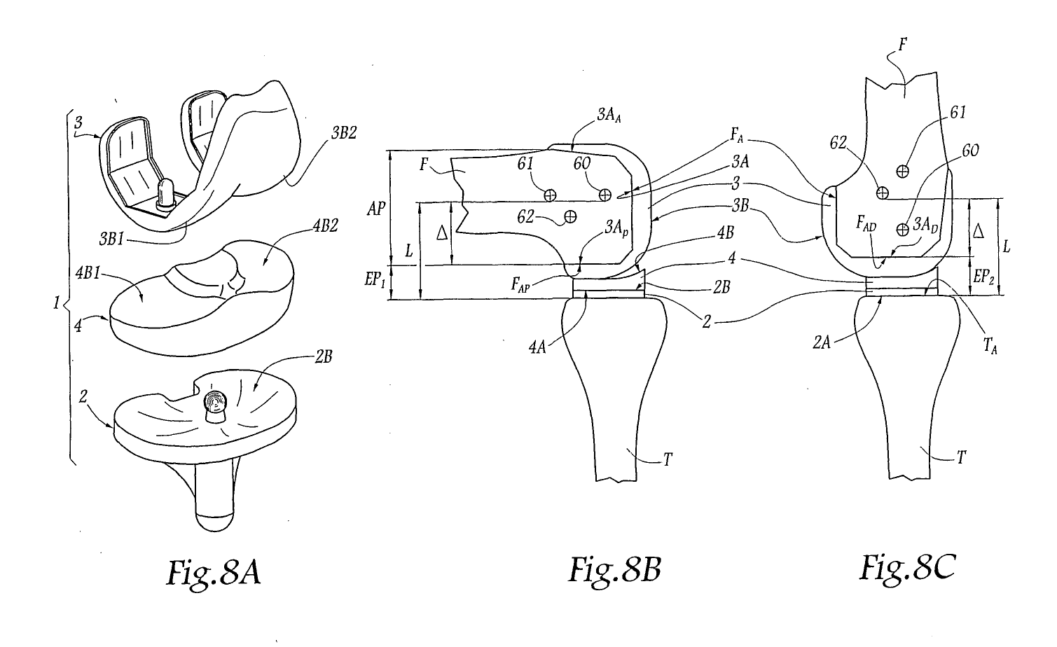

FIG. 8A shows an example 1 of such a “moving-plate” knee prosthesis. In known manner, the prosthesis 1 comprises a tibial component in the form of a metal base 2, a femur component in the form of a metal cap 3, and a polyethylene insert 4 constituting the moving plate of the prosthesis. As shown in FIGS. 8B and 8C, the tibial base 2 is designed to be secured firmly to the top end of the tibia T, and for this purpose it defines a bearing face 2A for bearing against and being secured to a corresponding resected surface T_{A} of the tibia, while the femoral cap 3 is firmly secured to the bottom end of the femur F, and for this purpose it defines a multi-plane face 3A for bearing against and being secured to a corresponding resected face F_{A} of the femur. The face 4A of the plate 4 that faces towards the base 2 rests movably on the top face 2B of said base, while the opposite face 4B of the plate defines two concave surfaces 4B₁ and 4B₂ designed to receive in hinged manner two associated convex surfaces 3B₁ and 3B₂ defined by the corresponding face 3B of the cap 3 and approximately reproducing the shape of the medial and lateral anatomical condyles of the femur.

The prosthesis 1 serves to reproduce movements close to those of the anatomical knee, thus guaranteeing good comfort in use for the patient. However the freedoms of movement internal to said prosthesis require the components of the prosthesis to be implanted with great accuracy and make it essential to take account of the ligamentary environment of the knee. In particular, in order to provide the best possible amplitudes of joint movement and the best possible stability, the surgeon seeks as much as possible to ensure that, in use, the values of the spacing between the femur and the tibia, both in flexion, in particular at 90°, and in extension, are equal. In practice, this spacing corresponds to the room made available to the portions of the prosthesis while they are in use, i.e. the portions of the prosthesis that come into hinged contact one against the other and that are of dimensions such as to present a total thickness in the direction concerned that is substantially identical both in flexion at 90° and in extension. This spacing is thus commonly referred to as the “prosthesis spacing”. Such prosthesis spacings in flexion and in extension are referenced EP₁ and EP₂ respectively in FIGS. 8B and 8C, the prosthesis spacing EP₁ in flexion being substantially equal to the distance between the resected tibial surface T_{A} and the plane of the position cut F_{AP} of the resected tibial face F_{A}, while the prosthesis spacing EP₂ in extension is substantially equal to the distance between the same tibial surface T_{A} and the plane of the distal cut F_{AD} of the multi-plane face F_{A} of the femur.

In addition, in order to avoid any excess pressure on one or other of the medial and lateral sides of the moving plate 4, or even dislocation of said plate, the ligamentary stresses, and in particular those due to the side ligaments, must be balanced both on the medial side and on the lateral side of the knee prosthesis.

In order to implant a knee prosthesis such as the prosthesis 1, the surgeon makes use successively and/or simultaneously of a plurality of different instruments. For example amongst these instruments, mention can be made of an instrument for performing resection of the top end of the tibia T.

Similarly, it is known to use a rod inserted in the medullary canal of the femur going from its bottom end. Manipulating this rod makes it possible, prior to securing the cap on the femur, to verify that the distal cut is perpendicular to the mechanical axis of the femur as identified by a pre-operative X-ray that is not always very accurate, for example because of a flexion deformity of the patient. However, it will be understood that such an inspection rod can only be inserted and moved while the knee is bent, i.e. when the bottom end of the femur is accessible along the axis of the femur. Thus, such an intramedullary rod does not enable ligamentary equilibrium to be verified in extension. When it comes to comparing the prosthesis spacings in flexion and in extension, this is assessed approximately by the surgeon. Furthermore, inserting the intramedullary rod generally leads to post-operative bleeding and increases the risk of infection.

In order to quantify ligamentary tension in extension, it is known to make use of another instrument, for putting the knee joint under tension, and commonly referred to as a distractor instrument. That instrument is generally in the form of two branches that are hinged relative to each other with distal ends that are provided with respective means for pressing against the tibia and for pressing against the condyles. By interposing, between the proximal portions of the branches, a mechanism for moving said portions apart, it is possible, after inserting the distal ends of said branches in the space between the femur and the tibia, to apply tension to the knee joint, in particular when it is in extension. For example by using differential dynamometric measurement means between the two femur condyles, the surgeon can verify that no significant ligamentary unbalance is present. Nevertheless, that type of distractor instrument is bulky and complicated to manipulate, thereby generally discouraging the surgeon who needs to manipulate in succession, and where necessary several times over, the above-mentioned intramedullary rod and the distractor instrument, thereby lengthening the time required for the operation.

The object of the present invention is to propose a set of instruments for implanting a knee prosthesis, which set of instruments is easy and fast to use, in particular because of the small number of instruments that need to be manipulated, and improves the accuracy with which the prosthesis is implanted, in particular concerning the space available for the prosthesis and concerning lateral ligamentary equilibrium, both in flexion at 90° and in extension.

To this end, the invention provides a set of instruments for implanting a knee prosthesis, the set including amongst others, a first instrument for distracting the knee, which instrument comprises firstly two branches movable relative to each other and respectively provided at their distal ends with means for pressing against the tibia and with means for pressing against the femur, and secondly means for moving the distal ends of the branches mutually apart, the set being characterized in that the means for pressing against the tibia define a tibial bearing surface that is substantially plane, and in that the distraction instrument is further provided with a device serving to locate, on at least one of the medial and lateral sides of the femur, the position for implanting at least one extramedullary pin or the like in a direction lying in a plane that is substantially parallel to the plane tibial bearing surface and that is situated at an adjustable distance from the tibial bearing surface.

With the single distraction instrument, the surgeon can verify medial/lateral ligamentary equilibrium of the knee both in flexion at 90° and in extension, and can automatically impose equal spacings for the prosthesis both in flexion and in extension. Firstly, the distal ends of the branches of the instrument can be inserted in the implantation space between the femur and the tibia so as to distract the knee, both in flexion and in extension, and secondly the locator device makes it possible on either side of the femur, on the medial side and on the lateral side, to implant in adjusted manner, e.g. at least one pair of extramedullary pins, and in practice several pairs and preferably at least three pairs, which pins serve to constitute markers in three dimensions for subsequent cutting of the bottom end of the femur, in particular for making distal and posterior cuts therein. By conserving the same adjustment for the spacing between the tibial resection face and firstly a first implantation of the extramedullary pins while the knee is flexed at 90°, and secondly a second implantation of extramedullary pins while the knee is in extension, the surgeon has markers for defining where to cut the femur that are suitable for guaranteeing equal spacings for the prosthesis in flexion and in extension. The plane containing the pins implanted while the knee is in flexion is substantially perpendicular to the axis of ligamentary tension in flexion and substantially parallel to the axis of the anterior cortex of the femur, while the plane containing the pins implanted while the knee is in extension is substantially perpendicular to the axis of ligamentary tension in extension.

According to other characteristics of this set of instruments, taken in isolation or in any technically feasible combination:

the locator device is suitable, both on the medial side and on the lateral side of the femur, for locating the positions for implanting at least one pair of extramedullary pins or the like along respective directions lying in a common plane that is substantially parallel to the plane tibial surface and that is situated at an adjustable distance from the bearing surface;

the locator device is suitable, on a given side of the femur, for locating the positions for implanting two extramedullary pins or the like in respective directions lying in a common plane substantially parallel to the plane tibial bearing surface;

the locator device includes extra-femoral jig means for defining the directions along which the pins or the like are implanted, these jig means defining, for example, extramedullary guide holes for means for forming cavities for receiving the pins or the like;

the distraction instrument includes a rod secured to the branch provided with the means for pressing against the tibia, and extending lengthwise along a direction that is substantially perpendicular to the plane containing the plane tibial bearing surface, and the locator device includes moving connection means between the rod and the jig means;

the moving connection means comprise first means for moving the jig means relative to the rod in translation along the rod, and the locator device includes adjustment and locking means for adjusting and locking the position in translation of the jig means;

the distraction instrument is provided with means for measuring the position in translation of the jig means relative to the rod, e.g. in the form of graduations carried by the rod;

the adjustment means comprise a feeler for feeling the anterior cortex of the femur;

the moving connection means comprise second means for moving the jig means relative to the rod in pivoting about the longitudinal axis of the rod;

the moving connection means include third means for moving the jig means relative to the rod in two directions that are substantially perpendicular to the longitudinal direction of the rod and substantially perpendicular to each other;

the set includes an extramedullary sight part for sighting the head of the femur and adapted to extend substantially parallel to the longitudinal direction of the rod;

the means for pressing against the femur define a convex elongate surface for pressing against the femur between the condyles thereof, and having a transverse dimension that is preferably less than about 9 (millimeters);

the set includes a second instrument for cutting the femur, fitted with means for positioning the instrument relative to the femur, that are adapted to co-operate with projecting portions of pins or the like implanted in the femur along directions defined by the locator device of the distraction instrument, the projecting portions projecting from the medial and lateral sides of the femur; and

the cutter instrument defines at least a plane posterior cutting slot and a plane distal cutting slot, and the positioning means comprise both a first pair of bearing surfaces for bearing against some of the pins or the like, substantially parallel to the posterior cutting slot, and a second pair of bearing surfaces for bearing against others of the pins or the like, substantially parallel to the distal cutting slot, the distance between the first pair of bearing surfaces and the plane of the posterior cutting slot being substantially equal to the distance between the second pair of bearing surfaces and the plane of the distal cutting slot.

The invention also provides a method for implanting a knee prosthesis, the method comprising the following successive steps:

resecting the top end of the tibia;

using a distraction instrument to distract the knee joint in bending at 90° and to verify ligamentary equilibrium between the medial and lateral sides of the knee, in particular by visually comparing the medial and lateral spaces behind the condyles;

using the same distraction instrument to distract the knee joint in extension and verifying ligamentary equilibrium between the medial and lateral sides of the knee, in particular by using an extramedullary sight part for sighting the head of the femur, and fitted on said instrument;

using a locator device carried by the distraction instrument, and while the knee in flexion is distracted by said instrument, locating at least on one of the medial and lateral sides of the femur the position for implanting at least one extramedullary pin or the like in a direction lying in a plane substantially parallel to the tibial resection plane, substantially perpendicular to the axis of ligamentary tension and situated at a given distance from said surface, which distance is adjusted relative to the position of the anterior cortex of the femur;

using the same locator device carried by the instrument with the knee in extension and distracted by said instrument to locate at least on one of the medial and lateral sides of the femur the position for implanting at least one extramedullary pin or the like in a direction lying in a plane substantially parallel to the tibial resection plane, substantially perpendicular to the axis of ligamentary tension and situated at said adjusted distance from said surface;

positioning a cutter instrument on the femur by co-operation between said cutter instrument and projecting portions of the extramedullary pins implanted in the femur in the previously-located positions, and portions projecting from the medial and lateral sides of the femur; and

making cuts in the femur, in particular a distal cut and a posterior cut.

Advantageously, the steps of verifying ligamentary equilibrium in flexion and in extension, and the steps of locating the implantation positions in flexion and in extension are performed with the kneecap of the patient generally in place.

The invention can be better understood on reading the following description given purely by way of example and made with reference to the drawings, in which:

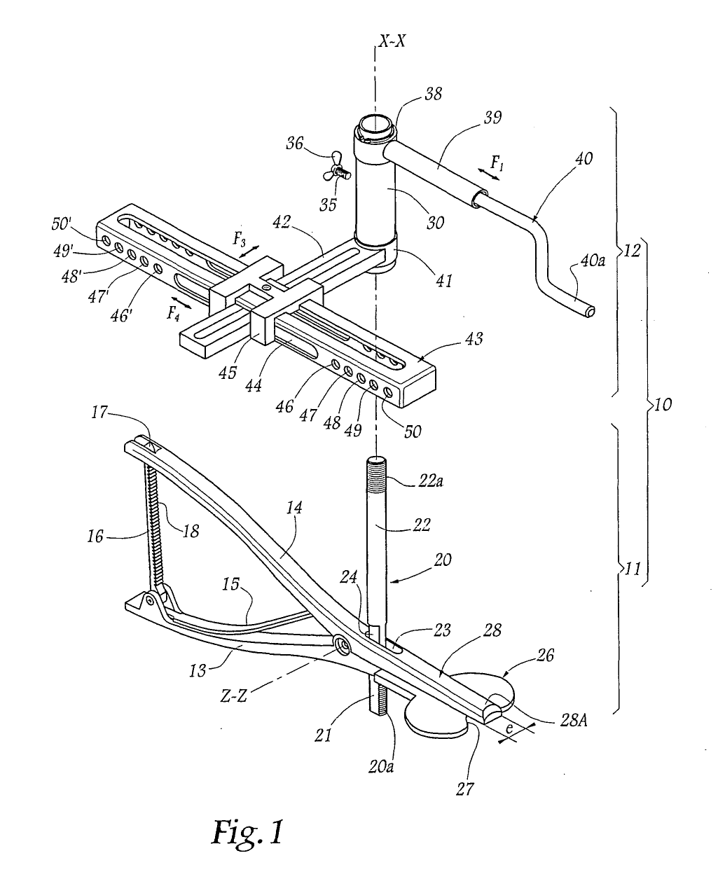

FIG. 1 is an exploded perspective view of a distraction instrument belonging to a set of instruments of the invention;

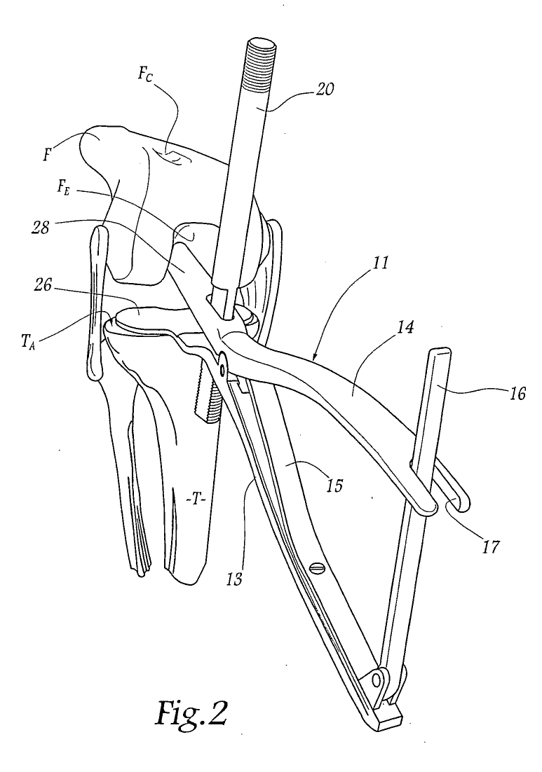

FIG. 2 is a perspective view showing a knee in flexion at 90° being distracted by the instrument of FIG. 1;

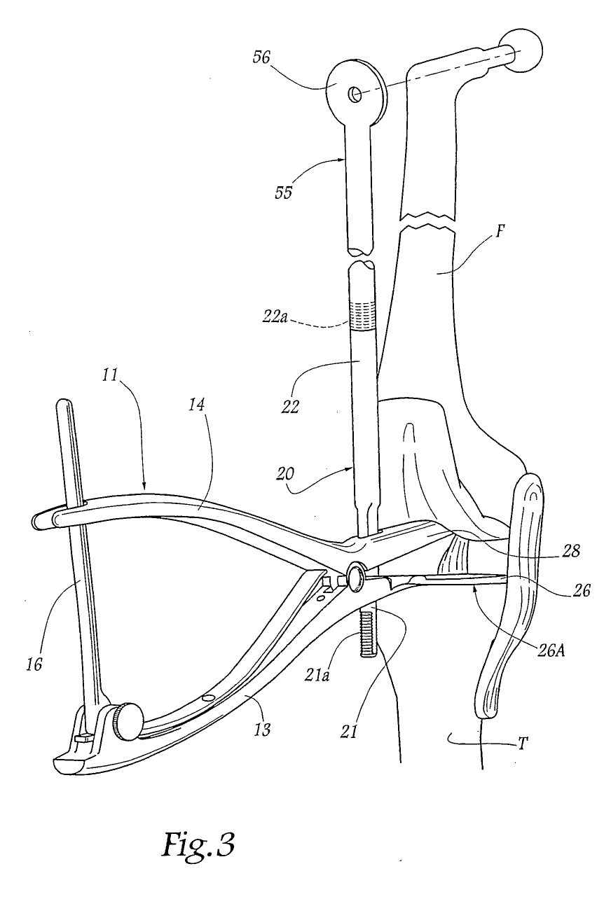

FIG. 3 is an analogous view to FIG. 2 showing the same knee in extension and being distracted by the instrument of FIG. 1;

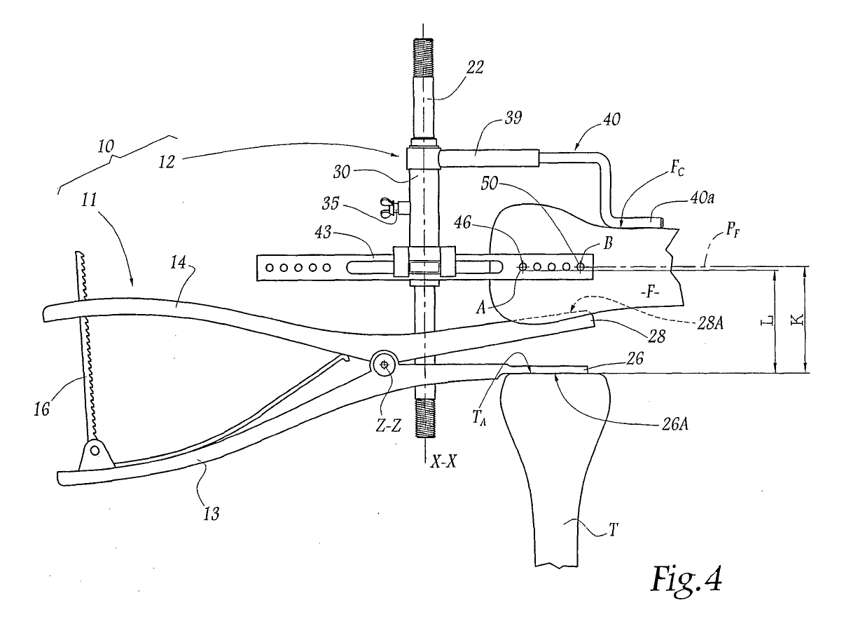

FIG. 4 is an elevation view of the FIG. 1 instrument while in use on the knee in flexion, in order to identify where extramedullary femoral pins are to be implanted;

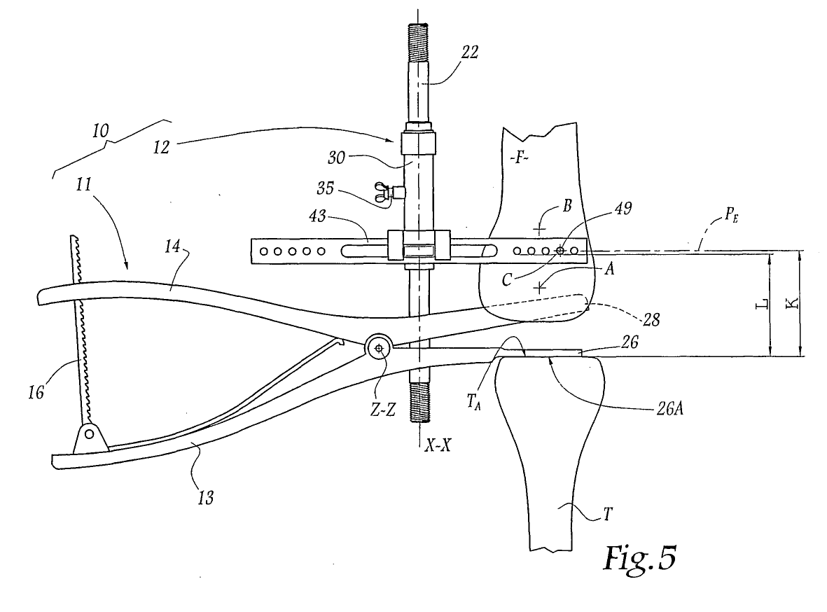

FIG. 5 is a view analogous to FIG. 4 showing the use of the FIG. 1 instrument on the knee in extension, in order to identify where other extramedullary femoral pins are to be implanted;

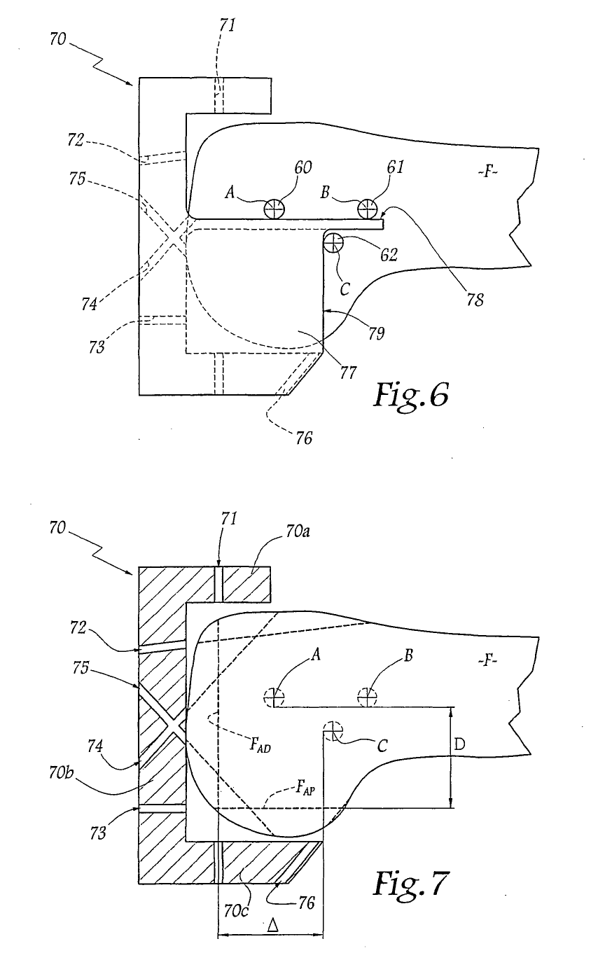

FIG. 6 is an elevation view of a femur-cutter instrument belonging to the set of instruments of the invention, positioned on the femur fitted with pins implanted in the positions identified in FIGS. 4 and 5;

FIG. 7 is a section view of FIG. 6 on a substantially median sagittal plane through one of the condyles of the femur;

FIG. 8A is an exploded perspective view of a knee prosthesis having a moving plate; and

FIGS. 8B and 8C are elevation views seen internally of the knee provided with the prosthesis of FIG. 8A, respectively in flexion and in extension.

FIG. 1 shows an instrument 10 for distracting the knee, for use in implanting the prosthesis 1 of FIG. 8A. This instrument 10 essentially comprises a main distractor device 11 and a locator device 12 suitable for being removably fitted on the main device 12, as explained below in detail.

The main device 11 comprises two elongate rigid branches 13 and 14 presenting a shape that is curved along their length. These branches are hinged to pivot relative to each other about an axis Z-Z extending substantially perpendicularly to the longitudinal direction of the branches. Between the proximal portions of the branches, i.e. their portions that face towards the surgeon while the device 11 is in use, there is interposed a spring blade 15 dimensioned to press continuously against the branches so their proximal portions move apart from each other, in other words so that their distal portions move towards each other. In order to enable the proximal portions of the branches to be adjusted and held in a close-together configuration, i.e. a configuration in which the distal portions of the branches are spaced apart, the device 11 is fitted with a rigid blade 16. The bottom end of the blade 16 is secured by a pin to the bottom branch 13, while its top end is received in a corresponding longitudinal notch 17 in the top branch 14. The distal face of the blade 16 is shaped to constitute a rack having teeth 18 that are adapted to engage the distal end of the notch 17.

In its distal portion, the bottom branch 13 is secured to a generally cylindrical rigid rod 20 having a circular base of axis X-X and extending substantially perpendicularly both to the longitudinal direction of said branch and to the axis Z-Z. By way of example, this rod 20 is welded to the branch 13, or else it is formed integrally together therewith.

The rod 20 has a bottom portion 21 that projects downwards from the bottom branch 13, and a top portion 22 that projects upwards from said branch and that presents a length that is longer than the bottom portion 21. The top portion 22 passes through the digital portion of the top branch 14 via an oblong hole 23 presenting a long dimension that extends along the branch 13 and level with which the rod portion 22 presents two corresponding diametrically-opposite flats 24 occupying planes that are substantially perpendicular to the axis Z-Z. The distance between the flats 24 is substantially equal to the short dimension of the hole 23, thereby preventing any significant transverse movement of the rod relative to the branch 14.

At its distal end, the bottom branch 13 is secured to a metal vane 26, e.g. formed integrally with the remainder of the branch. This vane is in the form of a plate that is plane, rigid, and small in thickness relative to the remainder of the branch. It thus defines a plane bottom face 26A lying in a plane that is substantially perpendicular to the axis X-X for the purpose of coming to bear against the resected surface T_{A} of the tibia T. For this purpose, the peripheral outline of the vane 26 is shaped so as to match as closely as possible the usual shape of a tibial resection, in particular by including a distal notch 27 designed to receive the posterior ligaments of the knee joint while the vane is being put into place in the space between the femur and the tibia.

At its distal end, the top branch 14 is in the form of a rigid finger 28 made integrally with the remainder of the branch, extending it longitudinally. The bottom surface of this finger is substantially plane, thereby providing plane-on-plane contact with the top face of the vane 26 when the proximal portions of the branches 13 and 14 are maximally spaced apart as shown in FIG. 1.

The top surface 28A of the finger 28 is convex in shape and is dimensioned so as to be suitable for being received in the anatomical notch between the condyles of the femur, as explained below. By way of example, this surface is semicylindrical and its width e is less than about 9 mm.

As mentioned above, the locator device 12 is adapted to be fitted removably on the device 11, and more precisely around its rods 20. This device 12 includes a tubular sleeve 30 of inside diameter substantially equal to the outside diameter of the top portion 22 of the rod 20. The sleeve 30 is thus fitted round the rod portion 22 so as to be movable both in pivoting about the axis X-X and in translation along said axis. To prevent the sleeve from moving relative to the rod, a lock screw 35 is provided to presses against the outside face of the rod 20 when screwed into a radial bore of the sleeve 30. The head of this screw 35 is advantageously provided with wings 36.

At its top end, the sleeve 30 is surrounded by an outer ring 37 that is free to move in rotation and that is prevented from moving in translation relative to the sleeve. Projecting radially from the ring there is a tube 39 made integrally with the ring, and receiving a bent rod 40. The portion of the rod 40 that is inserted in the tube 39 is free to move in translation inside the tube, as represented by double-headed arrow F₁. This tube portion defines a flat that is designed to extend facing a radial orifice (not shown) defined in the tube 39. A lock screw can be inserted into the orifice in order to lock the rod 40 relative to the tube so that the axis of its bent portion 40a extends perpendicularly to the axis X-X.

At its bottom end, the sleeve 30 is surrounded by another outer ring 41, likewise free to move in rotation and prevented from moving in translation relative to the sleeve. A rail 42 formed internally with the ring projects radially therefrom. A jig bar 43 is mounted to slide freely lengthwise on the rail 42, as represented by double-headed arrow F₃ in FIG. 1. More precisely, the bar defines a through slot 44 of oblong shape extending along its length, with the rail 42 being received therein. The length of the slot 44 is determined so as to allow the bar 43 to move in translation relative to the rail 42 along the longitudinal direction of the bar, as represented by double-headed arrow F₄, i.e. in a direction that is substantially at right angles to a radius relative to the axis X-X. In order to prevent the bar 43 from disengaging completely from the rail 42, and in order to stiffen the connection between the rail and the bar, a clearance-tightening bracket 45 is provided. A lock screw (not shown) is fitted to the bracket for the purpose of temporarily preventing the rail 42 and the bar 43 from moving relative to each other.

In each end portion of the bar 43 there are formed through holes 46 to 50 and 46′ to 50′ that are distributed along the length of the bar. The holes 46 to 50 are symmetrical to the holes 46′ to 50′ about the midplane of the bar 43 that is parallel to the axis X-X, the holes 46 and 46′ being the two holes that are situated closest to the slot 44, beside each of its ends. The respective axes of these holes 46 to 50 and 46′ to 50′ extend across the longitudinal direction of the bar 43 and all lie in a common plane that is substantially perpendicular to the axis X-X when the sleeve 30 is fitted on the rod 20.

The use of the distraction instrument 10 shown in FIG. 1 for the purpose of implanting the prosthesis 1 of FIG. 8A is described below with reference to FIGS. 2 to 5.

After cutting the knee zone of a patient to be operated and by way of example taking into consideration the preferred approach path which is internal, the surgeon resects the top end of the tibia T, e.g. by means of a conventional tibia-cutter instrument. At the end of this prior step, the top end of the tibia T presents the substantially plane resected surface T_{A} that is in principle substantially perpendicular to the mechanical axis of the tibia passing through the-central zone of said surface and the corresponding ankle of the patient. The bottom end of the femur F is, for the time being, intact.

In a first stage, the surgeon engages the distal ends of the branches 13 and 14 of the device 11 inside the space between the femur and the tibia. More precisely, and as shown in FIG. 2, the bottom face 26A of the vane 26 is pressed against the resected surface T_{A} of the tibia T, while the finger 28 is slid in register with the notch F_{E} between the condyles of the femur F. With the knee in flexion at 90°, the surgeon moves the proximal portions of the branches 13 and 14 of the instrument 1 towards each other so that the finger 28 is received between the medial and lateral condyles of the femur and moves the femur F progressively away from the tibia T. When the tension for spacing apart the tibia and the femur corresponds to an anatomically appropriate tension, the surgeon locks the top branch 14 relative to the bottom branch 13 by means of one of the teeth 18 of the blade 16. The device 11 of the instrument 10 is then in the configuration shown in FIG. 2.

The surgeon then has access to the spaces behind the condyles of the joint, and if necessary resects the posterior femoral and/or tibia osteophytes, and/or detaches the lateral ligaments from the flanks of the condyles and the condyle involucrums. When the surgeon can see that one of the spaces behind the condyles is larger than the other, which means that a difference in ligament tension remains between the sides of the knee joint, the surgeon proceeds to perform the appropriate surgical corrections, in particular by eliminating additional osteophytes and/or by more extensive detachment of ligaments.

It should be observed that it is necessary to dislocate the patient's kneecap in order to observe the spaces behind the condyles. Nevertheless, once the necessary surgical corrections have been performed, the kneecap can be reduced, the presence of the tapering finger 28 at the end of the top branch 14 not impeding the kneecap being put back into place. Equilibrium between the ligaments is thus controlled without significantly disturbing the ligamentary environment of the knee.

Furthermore, if before the operation there was a subluxation of the patient's kneecap, generally a lateral subluxation, then the surgeon might decide to remove more posterior condyle bone material medially rather than laterally while maintaining equal amounts of space behind the lateral and medial condyles. When it is subsequently implanted, the femur cap 3 of the prosthesis 1 will then be slightly offset laterally in rotation so as to be centered relative to the patient's kneecap.

In a second stage, once the equilibrium of the ligaments in flexion has been controlled in this way, the surgeon relaxes the distraction instrument 10 and brings the knee joint into extension, as shown in FIG. 3. As before, the surgeon then distracts the joint by moving apart the distal ends of the branches 13 and 14 until the joint is subjected to a distraction stress that substantially opposes the anatomical ligamentary tension. The surgeon then fits to the top end of the rod 20 a femoral sighting rod 55. By way of example, the rod 55 is screwed onto a corresponding thread 22a of the rod portion 22. Sight means 56 for sighting the anatomical head of the femur F are provided at the free end of the rod 55. If these sight means 56 sight the center of the head of the femur, e.g. as identified by a per-operative X-ray, then the surgeon concludes that the mechanical axis of the femur, i.e. the axis passing through said center and the zone FE between the condyles of the femur, coincides substantially with the ligamentary traction axis in extension. Otherwise, the surgeon resects any posterior osteophytes that are impeding the condyle involucrums of the knee.

In optional manner (not shown), the surgeon can verify that the mechanical axis of the femur and the mechanical axis of the tibia do indeed coincide substantially by fitting an additional tibial sighting rod on the device 11. This additional rod is, for example, screwed at one end around a corresponding thread 21a of the rod portion 21, while its opposite end extends to the patient's ankle.

As in the initial stages of the operation, once the joint has been distracted, the kneecap is reduced while verifying ligamentary equilibrium.

During a third stage, once ligamentary equilibrium in extension has been obtained as described above, the distraction instrument 10 is relaxed and the knee is brought back to the 90° bend configuration. As during the step of controlling ligamentary equilibrium in flexion, the branches 13 and 14 of the instrument 10 are used to distract the knee joint until the intensity of the distraction is substantially opposed to the anatomical ligamentary tension. The locator device 12, which has so far not been fitted to the device 11, is now put into place around the rod 20. The tube 39 of the top ring 38 is moved to extend substantially longitudinally in line with the femur, while the sleeve 30 is moved downwards in translation along the rod 20 until the bent portion 40a of the rod 40, now acting as a feeler, comes into contact with the anterior cortex F_{C} of the femur F, as shown in FIG. 4, where it is prevented from moving. It should be observed that the bent shape of the rod 40 makes it possible to pass over the enlargement of the epiphysis of the femur without being impeded thereby. The sleeve 30 is then prevented from moving in translation along the rod 20 by the lock screw 35. Thereafter, the bar 43 is moved initially in pivoting relative to the sleeve 30 so that one of its end portions, e.g. the end portion having the holes 46 to 50, is placed facing the medial flank of the femur, and is then moved in translation relative to the rail 42 so that the hole 46 is situated in the anterior-posterior direction a few millimeters away from the distal end face of the femur F. The distance that then exists between the bottom edge of the hole 46 and the tibial bearing surface 26A is referenced L in FIG. 4.

The surgeon then inserts a drill bit through the hole 46 so as to drill an extramedullary cavity in the bone on an axis referenced A and to a depth of several tens of millimeters. The surgeon also drills another cavity on an axis B by inserting the drill bit in one of the other holes 47 to 50 of the jig bar 43, e.g. the hole 50 of FIG. 4, so as to drill into the enlarged epiphysis of the femur rather than into its more-compact shaft. In each of the extramedullary cavities formed in this way, the surgeon implants a cylindrical pin (given respective references 60 and 61 in FIG. 6), each time leaving a portion of the pin projecting.

While retaining the positioning in translation of the sleeve 30 on the rod 20, the ring 41 is turned about the axis X-X so as to bring the other end portion of the jig bar 43 against the lateral flank of the femur F. Two other cavities are then drilled in the epiphysis of the femur, with the drill bit being guided respectively by the holes 46′ and 50′. Two other pins are then implanted in these extramedullary cavities, leaving respective portions projecting from the lateral side of the femur.

On each occasion the drill bit is being guided by one of the holes in the jig bar 43, the position in rotation of the ring 41 relative to the rod 20, and the position in translation of the bar 43 along the rail 42 can be adjusted so that the bar 43 is pressed as close as possible to the lateral flank of the femur F. As a result, the longitudinal axes A and B of the cavities formed are not necessarily parallel to one another. In contrast, these axes all lie in a common plane that is substantially parallel to the bearing surface 26A of the tibia, as represented by line P_{F} in FIG. 4 and situated relative to the surface 26A at a distance K associated with the adjustment of the position in translation of the sleeve 30 along the rod 20, and equal to the distance L plus the radius of the holes 46 to 50 and 46′ to 50′. This plane P_{F} is thus both parallel to the surface portion of the anterior cortex F_{C} of the femur F against which the bent portion 40a of the rod 40 has been put into contact in order to adjust the position in translation of the sleeve 30, and substantially perpendicular to the ligamentary traction axis in flexion because of the ligamentary equilibrium found in the first stage of the operation and because of the rotary movement of the notch F_{E} between the condyles on the surface 28a of the finger 28.

In a fourth stage, still without changing the position in translation of the sleeve 30 relative to the rod 20, the feeler rod 40 is pivoted, the main distraction device 11 is relaxed, the knee joint is put into extension, and then the device 11 is put back under tension so as to distract the joint, as during the second stage of the operation. As shown in FIG. 5, one of the holes in each of the series 46 to 50 and 46′ to 50′ in the bar 43 is then used in the same manner as before, i.e. for guiding the application of the above-mentioned drill bit so as to form extramedullary cavities in the medial and lateral sides of the femur F within which respective pins (referenced 62 in FIG. 6 for the medial flank) are implanted analogous to the pins mentioned above, with the pins including portions that project from the medial and lateral flanks of the femur. By way of example, in FIG. 5 it is the holes 49 and 49′ that are used and the axis of the cavity drilled in the medial side is referenced C. As before, the respective axes of the two extramedullary cavities (i.e. the medial and the lateral cavities) then lie in a plane that is substantially parallel to bearing surface 26A of the tibia, and situated at a distance K from said surface, as represented by the trace P_{E} in FIG. 5. This plane P_{E} is thus substantially perpendicular both to the mechanical axis of the femur and to the axis of ligamentary tension in extension because of the ligamentary equilibrium in extension found during the second stage of the operation and because of the movement in rotation of the notch F_{E} between the condyles on the surface 28a of the finger 28.

It should be observed that during the third and fourth stages of the operation, the kneecap is left in place or at most is offset very slightly laterally in order to allow the distal portions of the branches 13 and 14 to pass.

In a sixth stage, after withdrawing the instrument 10 and dislocating the kneecap, a cutter block 70 is positioned on the femur F as shown in FIGS. 6 and 7. This femur-cutter block 70 is adapted to guide femur-cutting blades so as to produce femur resection planes that match planes of the face 3A of the femur cap 3 that is to be implanted.

More precisely, in its main portion, the block 70 presents a section that is generally in the form of an open C-shape suitable for receiving the end of the femur F with the knee bent at 90°, as shown in FIG. 7. This block defines:

in its top branch 70a, a distal cutting slot 71;

in its base 70b, an anterior cutting slot 72 and a pair of posterior cutting slots 73 with each individual slot in this pair being associated with a respective condyle of the femur, and also angled cutting slots 74 and 75 for producing cuts that are inclined, respectively interconnecting the anterior and distal cuts and the posterior and distal cuts; and

in its bottom branch 70c, a pair of extra-posterior cutting slots 76 for forming condyle cuts leading to the posterior cuts that slope forwards relative thereto, e.g. at about 45°.

It should be observed that the distal and posterior slots 71 and 73 are substantially perpendicular relative to each other, such that the cuts made using these slots are those that are to extend substantially parallel to the resected surface T_{A} of the tibia T when the knee is respectively in extension (as in FIG. 8C), and in flexion (as in FIG. 8B).

The condyle cuts to be performed using the pair of slots 76 are not designed to correspond to a portion of the multi-plane face 3A of the cap 3, but are adapted to make it possible to eliminate any surplus bone that might be responsible for conflict between the posterior portion of the resected end F_{A} of the femur and the posterior edge of the moving plate 4 when the knee with the prosthesis fitted is in extreme flexion.

In order to obtain femur cuts on planes that are suitably positioned for implanting the prosthesis 1, the block 70 is provided with side flanks 77 that are to be placed on the medial and lateral sides of the femur and that are symmetrical about a midplane of the block, perpendicular to the posterior slot 73. Each flank 77 presents firstly a first plane bearing surface 78 parallel to the posterior slot 73 that extends along the mass of the femur epiphysis when the block is in position on the femur, and secondly a second plane bearing surface 79 that is substantially perpendicular to the surface 78, in other words substantially parallel to the distal slot 71. These surfaces 78 and 79 extend at the same distance Δ respectively from the plane of the posterior slot 73 and the plane of the distal slot 71. This distance Δ is predetermined as a function of the size of the femur cap 3 for implanting. In the example described, the distance Δ is equal to the distal AP (FIG. 8B) between the top end point of the anterior plane 3A_{A} of the multi-plane face 3A of the cap 3, and the posterior plane 3A_{P} of said multi-plane face 3A, minus a predetermined constant J that is a function of the fixed dimensions of the device 12. The constant J is equal to the distance along the axis X-X between the bottom face 40a of the feeler and the plane containing the axes of the holes 46 to 50 and 46′ to 50′, plus the radius of said holes. Thus, for each size of femoral implant, i.e. for each distance AP, there is an associated cutter block analogous to the block 70. In use, as shown in FIGS. 6 and 7, the cutter block 70 is positioned on the femur F in such a manner that the surface 78 is pressed against the pins 60 and 61, while the surface 79 is pressed against the pins 62.

Once this positioning has been achieved, the cutter block 70 is, if necessary, prevented from moving relative to the femur, e.g. by means of handles or pegs (not shown) for preventing it from moving, that pass through each flank 77 of the instrument, and that are anchored in the bone. In an option that is not shown, each flank 77 is fitted with a bar parallel to the corresponding surface 78 and movable in a direction that extends transversely relative to said surface. This bar can thus be moved relative to the surface 78 so as to be clamped against the pins 60 and 61, and means for locking the bar are provided to prevent it from moving. The cutter block is thus stabilized to a greater extent on the femur, so as to limit as much as possible the use of other movement-preventing means that would be invasive.

Cutter blades or saw blades are then inserted in the various slots of the instrument. In particular, inserting such a blade in the posterior slot 73 enables posterior resection to be performed on the femur F in the cutting plane F_{AP} that is located at a distance Δ from the pins 60 and 61, and inserting the blade in the slot 71 enables distal resection to be performed on the femur in the cutting plane F_{AD} located at a distance Δ from the pin 62. Consequently, firstly the distance between the plane of the posterior cut F_{AP} and the tibial surface T_{A} is equal to L−Δ when the knee is flexed to 90° as in FIG. 8B, and secondly the distance between the distal cutting plane F_{AD} and said tibial surface T_{A} is likewise equal to L−Δ when the knee is in extension, as in FIG. 8C. In other words, the spaces occupied by the prosthesis in flexion EP₁ and in extension EP₂ as obtained by using the cutter block 70 positioned on the femur F by means of the pins 60 to 62 are substantially equal to each other and satisfy the relationship:
EP₁=EP₂=L−Δ=L−AP+J

The tibial plate 2 of the prosthesis 1 is then secured to the tibia, the moving plate 4 is put into place, and finally the femoral cap 3 is secured to the femur F.

The use of the two instruments 10 and 70 of the invention thus makes it possible to obtain quickly, easily, and accurately prosthetic compartments in flexion and in extension that are substantially equal in height. For example, only one assistant is needed beside the surgeon.

Both of the instruments 10 and 70 occupy only a small amount of space, and each of them can be used without difficulty by the surgeon, without significantly lengthening the duration of surgery, and possibly even shortening it.

In addition, setting up a ligamentary environment that is free from any excess tension or tension unbalance reduces the level of stresses between the metal components 2 and 3 and the polyethylene plate 4, or at least avoids forming zones that are over-stressed. Wear of the polyethylene is thus diminished, or in any event slowed. Delamination starters and the appearance of medial or lateral decooptation are-also avoided.

In a particularly advantageous arrangement of the invention (not shown), the top portion 22 of the rod 20 presents a series of graduations suitable for quantifying the position in translation of the sleeve 30 along said rod when it is prevented from moving by the lock screw 35. Using this measurement and knowing the size of the femoral component of the prosthesis that is to be implanted, the surgeon is in a position, e.g. by using a nomogram chart, to determine the thickness of the moving plate that should be implanted. Identifying the position of the sleeve along the rod 20 gives information representative of the above-defined distance L (or K), while the size selected for the tibial and femoral components of the prosthesis that is to be implanted gives in advance the value Δ associated with the prosthesis. As a result, it is possible to determine the spacing to be occupied by the prosthesis that results from taking the difference between the distance L and the value Δ, so by subtracting the thicknesses of the femoral cap 3 and the tibial base plate 2, it is possible to obtain the most appropriate value for the thickness of the plate 4, i.e. a thickness value that makes it possible to obtain a prosthesis of thickness substantially equal to the spacing available for the prosthesis in flexion and in extension. During surgery, the surgeon can thus decide which plate 4 is the most appropriate for the patient being operated.

Assuming that the surgeon does not have an almost continuous range of plate thicknesses, but rather a series of plates of thicknesses that increase gradually by steps of non-negligible size, it can happen, for example, that using the above-described method the surgeon decides that the thickness that is the most appropriate for the tibial plate to be implanted is about 9 mm whereas the available plates have respective thicknesses of 8 mm and 10 mm. Under such circumstances, it is advantageous in the invention to make provision for a plurality of types of pin to be implanted in the epiphysial mass of the femur. More precisely, in addition to cylindrical pins 60 to 62 of outside diameter that is substantially constant along their length, cylindrical pins are also available that are externally stepped, i.e. in which the portion for anchoring in the bone is of a diameter that is substantially equal to the diameter of the pins 60 to 62, while the opposite portion has an outside diameter that is slightly smaller and/or slightly greater than the outside diameter of the pins 60 to 62, e.g. by about 0.5 mm, 1 mm, or 1.5 mm. As a result, by implanting such stepped pins on the medial and lateral sides of the femur during the fifth stage of the operation, the surgeon can offset the position of the cutter block 70 by about 1 mm relative to the position it would otherwise occupy if the pins 60 to 62 were used as described above, such that one of the above-mentioned plates having a thickness of 8 mm or of 10 mm will end up being implanted while also adapting well to the spacing available for the prosthesis.

In a variant, instead of implanting pairs of pins as described above, the surgeon may implant pins on the medial or the lateral side only of the femur F, which pins are of a length that is sufficient to ensure that once implanted they project from both sides of the femur. Under such circumstances, greater damage is done to the epiphysis of the femur, but providing the holes drilled to receive the through pins are indeed straight, only one step of locating and implanting pins suffices on only one of the medial and lateral sides of the femur, thereby further shortening the duration of surgery.

In addition, whether the implanted pins are through pins or in pairs, provision can be made for them to be surrounded by a single-use sheet of plastics material so as to prevent them from being re-used.

Furthermore, although the instruments 10 and 70 are described above as being for the purpose of implanting a prosthesis 1 having a moving plate, the instruments can naturally also be used for implanting a knee prosthesis that does not have such a moving plate.

It is also possible to envisage various adaptations or variations to the instruments described above:

the distraction device 11 may be fitted with a system for declutching the spacer blade 16 so as to avoid harming knee ligaments in the event of over-tensioning;

similarly, dynamometric measurement means may be provided between the branches 13 and 14 to quantify the tension to which the knee is subjected;

the toothed blade 16 may be replaced by a threaded rod secured to the branch 13, parallel to the axis X-X and having a knurled wheel movable therealong, the wheel being associated with the branch 14 in the manner of a screw-and-nut system; under such circumstances, the extramedullary sight part 55 for sighting the head of the femur can be fitted to the free end of said threaded rod;

instead of the single jig bar 43 that can be turned about the axis X-X relative to the sleeve 30, the locator device 12 may have two jig bars that are stationary in rotation relative to the sleeve 30 and that are disposed on either side of the axis X-X; under such circumstances, the ring 41 can be made integrally with the sleeve 30, for example, and two rails 42 can be provided symmetrically on either side of the sleeve 30, each rail being slidably connected to a respective open of these two jig bars;

a circularly cylindrical ring may be mounted to turn freely about the free end of the finger 28 so as to make it easier to tilt the femur via its notch F_{E} between the condyles relative to the branch 14;

the distractor device 11 can be actuated in inverse manner, i.e. provision can be made for the distal ends of the branches 13 and 14, when at rest, to be spaced apart from each other by a compression spring, interposed between the proximal portions of said branches, so that by compressing the spring these distal portions are moved towards each other, thereby enabling them to be inserted in the space between the femur and the tibia; under such circumstances, the above-mentioned compression spring is dimensioned so that when the proximal portions of the branches of the instrument are released, the joint is tensioned to a value that is anatomically appropriate;

the instruments 10 and 70 may be adapted to computer-assisted navigation; and/or

the cutter block 70 is not necessarily made as a single piece; in particular, it is possible to use a pre-existing cutter sub-block provided with at least one posterior cutting slot, and to mount said sub-block rigidly on a frame having arrangements in accordance with the invention, and in particular having a distal cutting slot provided therein and having flanks that are analogous to the flanks 77 described above.

1. A set of instruments (10, 70) for implanting a knee prosthesis (1), the set including amongst others, a first instrument (10) for distracting the knee, which instrument comprises firstly two branches (13, 14) movable relative to each other and respectively provided at their distal ends with means (26) for pressing against the tibia and with means (28) for pressing against the femur, and secondly means (16) for moving the distal ends of the branches mutually apart, the set being characterized in that the means (26) for pressing against the tibia define a tibial bearing surface (26A) that is substantially plane, and in that the distraction instrument (10) is further provided with a device (12) serving to locate, on at least one of the medial and lateral sides of the femur (F), the position for implanting at least one extramedullary pin (60; 61; 62) or the like in a direction (A; B; C) lying in a plane (P_{F}; P_{E}) that is substantially parallel to the plane tibial bearing surface (26A) and that is situated at an adjustable distance (K) from said tibial bearing surface.

2. A set of instruments according to claim 1, characterized in that the locator device (12) is suitable, both on the medial side and on the lateral side of the femur (F), for locating the positions for implanting at least one pair of extramedullary pins (60; 61; 62) or the like along respective directions (A; B; C) lying in a common plane (P_{F}; P_{E}) that is substantially parallel to the plane tibial surface (26A) and that is situated at an adjustable distance (K) from said bearing surface.

3. A set of instruments according to claim 1, characterized in that the locator device (12) is suitable, on a given side of the femur, for locating the positions for implanting two extramedullary pins or the like (60, 61) in respective directions (A, B) lying in a common plane (P_{F}) substantially parallel to the plane tibial bearing surface (26A).

4. A set of instruments according to claim 1, characterized in that the locator device (12) includes extra-femoral jig means (43) for defining the directions (A; B; C) along which the pins (60; 61; 62) or the like are implanted, these jig means (43) defining, for example, extramedullary guide holes (46 to 50, 46′ to 50′) for means for forming cavities for receiving the pins or the like.

5. A set of instruments according to claim 4, characterized in that the distraction instrument (10) includes a rod (20) secured to the branch (13) provided with the means (26) for pressing against the tibia, and extending lengthwise along a direction (X-X) that is substantially perpendicular to the plane containing the plane tibial bearing surface (26A), and in that the locator device (12) includes moving connection means (30, 41, 42, 44, 45) between said rod and the jig means (43).

6. A set of instruments according to claim 5, characterized in that said moving connection means comprise first means (30) for moving the jig means (43) relative to the rod (20) in translation along said rod, and in that the locator device (12) includes adjustment and locking means (40, 36) for adjusting and locking the position in translation of the jig means.

7. A set of instruments according to claim 6, characterized in that the distraction instrument (10) is provided with means for measuring the position in translation of the jig means (43) relative to the rod (20), e.g. in the form of graduations carried by said rod.

8. A set of instruments according to claim 6, characterized in that said adjustment means (40) comprise a feeler (40a) for feeling the anterior cortex (F_{C}) of the femur (F).

9. A set of instruments according to claim 5, characterized in that the moving connection means comprise second means (41) for moving the jig means (43) relative to the rod (20) in pivoting about the longitudinal axis (X-X) of said rod.

10. A set of instruments according to claim 5, characterized in that the moving connection means include third means (42, 44) for moving the jig means (43) relative to the rod (20) in two directions (F₃, F₄) that are substantially perpendicular to the longitudinal direction (X-X) of the rod and substantially perpendicular to each other.

11. A set of instruments according to claim 5, characterized in that it includes an extramedullary sight part (55) for sighting the head of the femur and adapted to extend substantially parallel to the longitudinal direction (X-X) of the rod (20).

12. A set of instruments according to claim 1, characterized in that the means (28) for pressing against the femur define a convex elongate surface (28A) for pressing against the femur between the condyles thereof, and having a transverse dimension (e) that is preferably less than about 9 mm.

13. A set of instruments according to claim 1, characterized in that it includes a second instrument (70) for cutting the femur, fitted with means (78, 79) for positioning the instrument relative to the femur (F), that are adapted to co-operate with projecting portions of pins or the like (60 to 62) implanted in the femur along directions (A, B, C) defined by the locator device (12) of the distraction instrument (10), said projecting portions projecting from the medial and lateral sides of the femur (F).

14. A set of instruments according to claim 13, characterized in that the cutter instrument (70) defines at least a plane posterior cutting slot (73) and a plane distal cutting slot (71), and in that the positioning means comprise both a first pair of bearing surfaces (78) for bearing against some (60, 61) of said pins or the like (60 to 62), substantially parallel to the posterior cutting slot (73), and a second pair of bearing surfaces (79) for bearing against others (62) of said pins or the like, substantially parallel to the distal cutting slot (71), the distance (A) between said first pair of bearing surfaces (78) and the plane of the posterior cutting slot (73) being substantially equal to the distance (A) between said second pair of bearing surfaces (79) and the plane of the distal cutting slot (71).

15. A method for implanting a knee prosthesis (1), the method comprising the following successive steps:
resecting the top end of the tibia (T);
using a distraction instrument (10) to distract the knee joint in bending at 90° and to verify ligamentary equilibrium between the medial and lateral sides of the knee, in particular by visually comparing the medial and lateral spaces behind the condyles;
using the same distraction instrument (10) to distract the knee joint in extension and verifying ligamentary equilibrium between the medial and lateral sides of the knee, in particular by using an extramedullary sight part (55) for sighting the head of the femur, and fitted on said instrument;
using a locator device (12) carried by the distraction instrument (10), and while the knee in flexion is distracted by said instrument, locating at least on one of the medial and lateral sides of the femur (F) the position for implanting at least one extramedullary pin or the like (60; 61) in a direction (A; B) lying in a plane (P_{F}) substantially parallel to the tibial resection plane (T_{A}), substantially perpendicular to the axis of ligamentary tension and situated at a given distance (K) from said surface, which distance is adjusted relative to the position of the anterior cortex (F_{C}) of the femur (F);
using the same locator device (12) carried by the instrument (10) with the knee in extension and distracted by said instrument to locate at least on one of the medial and lateral sides of the femur (F) the position for implanting at least one extramedullary pin or the like (62) in a direction (C) lying in a plane (P_{E}) substantially parallel to the tibial resection plane (T_{A}), substantially perpendicular to the axis of ligamentary tension and situated at said adjusted distance (K) from said surface;
positioning a cutter instrument (70) on the femur (F) by co-operation between said cutter instrument and projecting portions of the extramedullary pins (60 to 62) implanted in the femur in the previously-located positions, and portions projecting from the medial and lateral sides of the femur (F); and
making cuts in the femur, in particular a distal cut (F_{AD}) and a posterior cut (F_{AP})

16. A method according to claim 15, wherein the steps of verifying ligamentary equilibrium in flexion and in extension, and the steps of locating the implantation positions in flexion and in extension are performed with the kneecap of the patient generally in place.
